(19) United States
(12) Patent Application Publication
(10) Pub. No.: US 2020/0015725 A1
(43) Pub. Date: Jan. 16, 2020

(54) CASSETTE FOR BLOOD SAMPLE MEASUREMENT COLLECTION AND STORAGE

(71) Applicant: Boston Microfluidics, Inc., Cambridge, MA (US)

(72) Inventors: Brandon T. Johnson, Cambridge, MA (US); Keith Kopitzke, Fallbrook, CA (US); Catherine Fink, Carlisle, MA (US)

(21) Appl. No.: 16/421,696

(22) Filed: May 24, 2019

Related U.S. Application Data
(63) Continuation-in-part of application No. 16/173,101, filed on Oct. 29, 2018.
(60) Provisional application No. 62/675,870, filed on May 24, 2018.
(60) Provisional application No. 62/715,476, filed on Aug. 7, 2018.
(60) Provisional application No. 62/820,411, filed on Mar. 19, 2019.

(51) Int. Cl. A61B 5/15

(52) U.S. Cl. CPC A61B 5/150343; A61B 5/150015; A61B 5/1405; A61B 5/150213; A61B 5/150755; A61B 5/150946

(57) ABSTRACT

A device uses push-initiated force to collect, meter, filter and store a blood sample. In one implementation, the push-initiated force may be provided in a cassette-type housing that also collects, meters, filters and/or stores the blood sample.

### CROSS REFERENCE TO RELATED APPLICATIONS

This application claims priority to a co-pending U.S. Provisional Patent Application Ser. No. 62/675,870 filed May 24, 2018 entitled “Push- Or Twist-Initiated Fluid Metering, Filtering and/or Storage”, a co-pending U.S. Provisional Application Ser. No. 62/715,476 filed Aug. 7, 2018 entitled “Push- Or Twist-Initiated Blood Metering, Filtering and/or Storage”, a co-pending U.S. Non-Provisional application Ser. No. 16/173,101 filed Oct. 29, 2018 entitled “Push- Or Twist-Initiated Blood Metering, Filtering and/or Storage”, and a co-pending U.S. Provisional Application Ser. No. 62/820,411 filed Mar. 19, 2019 entitled “Cassette for Blood Sample Measurement Collection and Storage”.

The entire contents of each of the above-referenced applications are hereby incorporated by reference.

### BACKGROUND

### Technical Field

This application relates to devices and methods for blood sample collection, metering, filtering and storage.

### Background Information

Blood used for diagnostic testing is usually extracted from a patient with a hypodermic needle and collected in a test tube. The collected blood is then packaged for shipment to a remote lab where various diagnostic tests are performed. However, many diagnostic tests require significantly less volume than the collected sample. Separation of cellular components from the sample is also needed for some tests.

Many tests only require small blood samples, where a finger stick rather than a hyperdermic needle can produce enough blood. But this small amount of blood cannot be easily transported to a lab. If the testing method cannot be immediately used at the same time the blood is extracted, a convenient reliable method of capturing, prepping, and preserving small amounts of blood is needed.

### SUMMARY

A device uses push-initiated force to collect, meter, filter and store a blood sample. The device includes a housing, a metering assembly, and blood storage media.

In some configurations, the housing is provided as a cassette-type arrangement, where hinged door provides a push-initiated force. In this arrangement, a cover may be provided to protect the device and prevent undesired activation or access during shipment. In this arrangement, a blood collection sub assembly includes one or more microfluidic channels to collect a blood sample and meter it. The channel(s) may collect blood from a sample port via a passive type wicking action or in other ways. The sample port may be an oval-shaped cup with an upper ridge that provides a surface on which the user may scrape their finger to encourage blood collection. A fill window coupled to the channel(s) at the far end enables the user to monitor the progress of sample collection.

A frame (or support) assembly includes a collection element such as LF1 paper or some other suitable membrane or media for storing the sample. The collection element may be treated with various agents or composed of different parts that dry, separate, filter, stabilize, treat, or analyze the sample in different ways. Notches on the frame may be provided to further align with ribs on a bottom housing.

A top housing piece may be provided with a hinged door that is activated after taking a sample. Two small, breakable bridges may hold the hinged door in place during assembly and before the sample is collected. A certain amount of force, such as a few pounds, breaks the bridges, permitting the hinged piece to snap down into a final horizontal position to close the device after use.

To collect a blood sample, the patient's pricked finger is scraped along an upper edge of the blood collection well permitting blood to drip down into the port below. As the port is filled, the blood flows into the microfluidic channels and eventually reaches the final fill window. After a sufficient blood sample is taken, the hinged door is snap-closed by the user. This closing action then also actually activates contact between the exit end of the microfluidic channels and the collection paper. In other words, the end of the channel where the blood has reached is initially lifted off of disposed and away from the collection paper. However, pressing down on the top housing breaks the bridge pieces so that the sample port now comes in contact with the edge of the collection paper, which then will initiate a wicking process of the blood into the paper.

### BRIEF DESCRIPTION OF THE DRAWINGS

The description below refers to the accompanying drawings, of which:

FIG. 1 is a perspective view of a cassette device that includes a hinged or sliding door that supplies a push-initiated force.

FIG. 2 is an exploded view the device shown in FIG. 1.

FIG. 3 shows a blood collection assembly in more detail.

FIG. 4 shows a support assembly.

FIG. 5 illustrates the device in an initial stage of being assembled.

FIG. 6 shows the top housing being assembled.

FIG. 7A shows the top housing facing downwards to engage pins into holes in the bottom piece.

FIG. 7B is a closer view of one implementation where a microfluidic subassembly may have thinned portions or perforations designed to break when a door is closed.

FIG. 8 shows the device being used to collect a blood sample.

FIGS. 9A and 9B show the door being closed.

FIGS. 10A and 10 B show a hinged port cover being closed over the device after the sample is taken.

FIGS. 11A and 11 B are alternate design for the port cover.

FIGS. 12A and 12 B are another design for the closure.

FIGS. 13 and 14 show how the device is disassembled to obtain access to the sample.

### DETAILED DESCRIPTION OF AN ILLUSTRATIVE EMBODIMENT

In one embodiment, the device is implemented as a cassette-type device that collects, meters and stores body fluids such as a blood sample. The cassette form factor includes a hinged or sliding door that provides the push-initiated force.

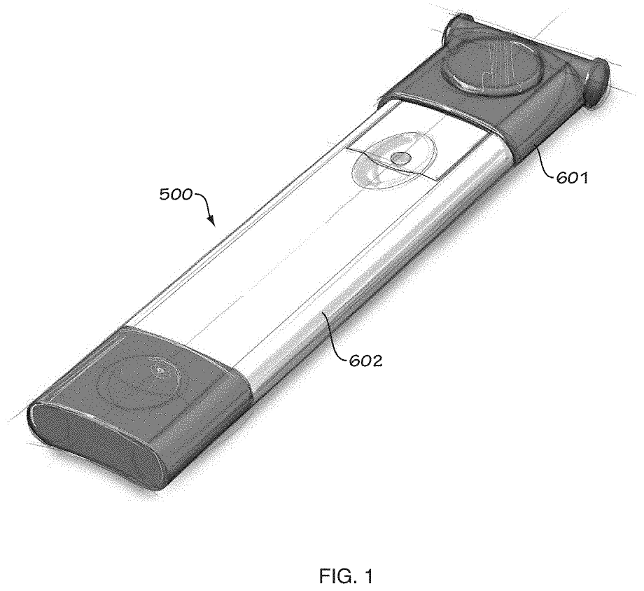

FIG. 1 is a perspective view of the assembled cassette device 500 which includes a cover 601 and housing 603.

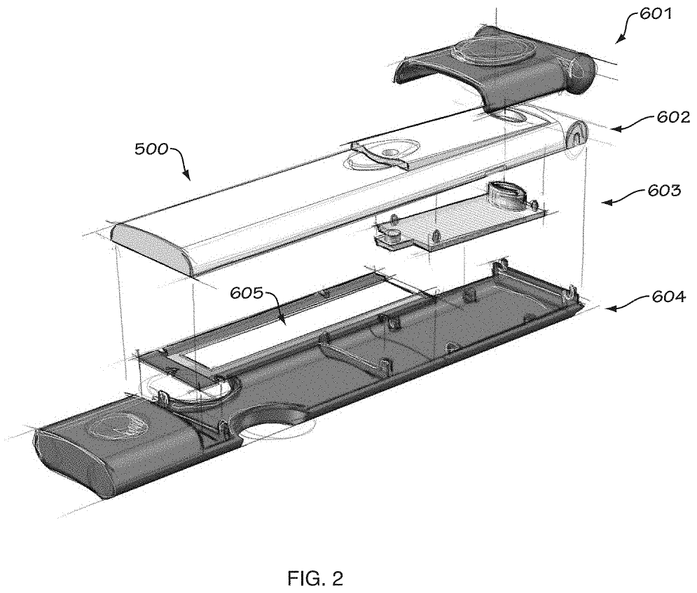

FIG. 2 is an exploded view of the cassette device 500 showing its five primary components more particularly, including a sample port cover 601, a top housing 602, a blood collection (or “chip”) assembly 603, bottom housing 604 and frame assembly 605.

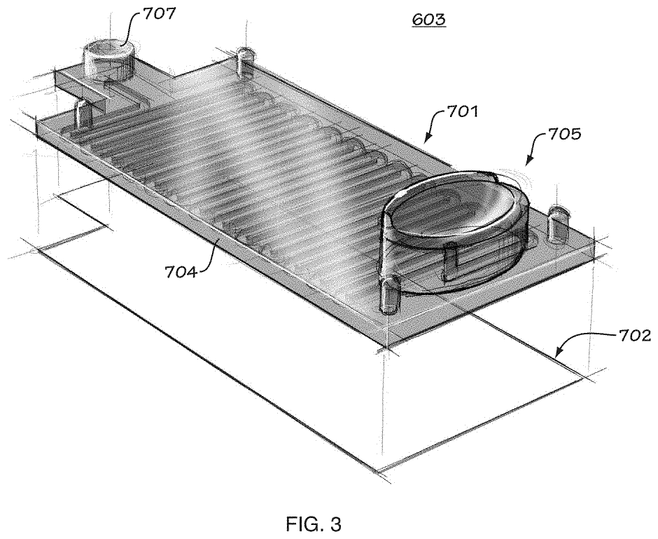

FIG. 3 shows the blood collection assembly 603 in more detail. It includes a blood collection chip 701 and some sort of a sealer or adhesive on the bottom such as a Pressure Sensitive Adhesive (PSA) 702. The blood collection chip includes one or more microfluidic channels 704 to collect a blood sample and meter it. In one implementation, the channel is a serpentine-shaped channel roughly a one millimeter in diameter and with a length to hold a sample of about 180-200 microliters. However other dimensions are possible. The channel 704 collects blood from a sample port 705 via a passive type wicking action from any fluid deposited in the sample port.

In this particular design, the sample port 705 is an oval-shaped cup with an upper ridge 706. The ridge provides a surface on which the user may scrape their finger to encourage blood collection. However other designs for the sample port are possible.

The microfluidic channel(s) 704 can be self-hydrophilic or in other implementations the channel(s) may be coated with anticoagulant.

A fill window 707 coupled to the channel(s) at the far end enables the user to monitor the progress of sample collection.

To ensure a metered amount of blood has been collected, a method of separating the microfluidic channel from the sample collection well may be implemented to avoid overfilling. This may include a channel that breaks away upon activation (such as by snapping the device closed, described in more detail below) or otherwise separates the fluid contact.

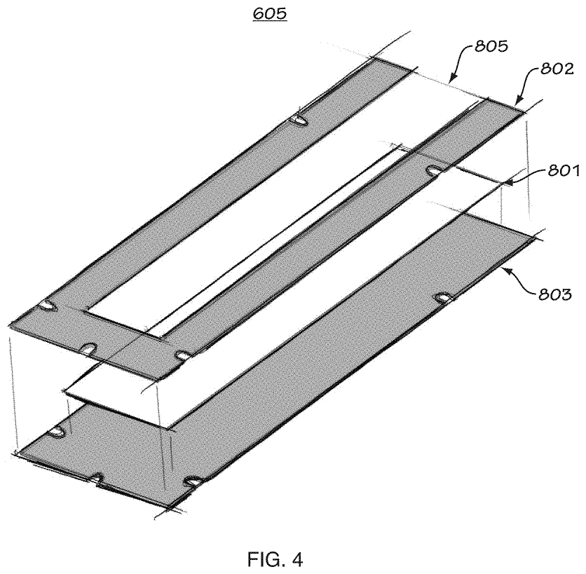

FIG. 4 shows the frame (or support) assembly 605 in more detail, including a collection element 801 such as LF1 paper, a top frame 802 and a bottom frame 803. Notches are formed on the outside edges and used for alignment and for assembly purposes. One end 805 of the top frame may be open.

The frame pieces 802, 803 may be formed from different color mylars to differentiate the frame pieces from the LF1 paper 801. The frame assembly 605 may be assembled on a high throughput machine where there will be adhesive to bonded the parts together. In that approach, the parts may be delivered as a roll of series connected pieces to ease mass production.

The collection element 801 may be LF1 paper or some other suitable membrane or media for storing the sample. The collection element may be treated with various agents or composed of different parts that dry, separate, filter, stabilize, treat, or analyze the sample in different ways.

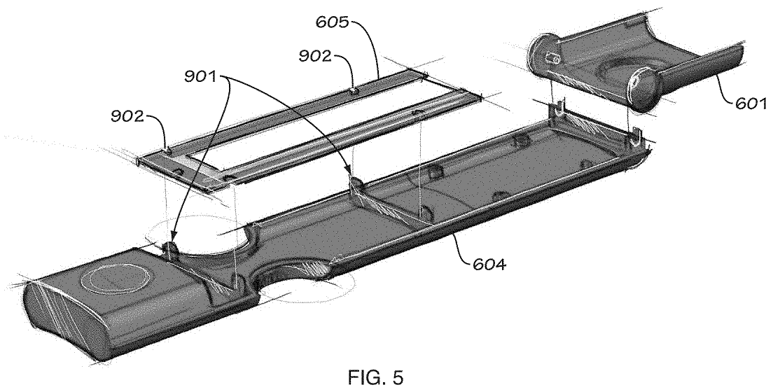

FIG. 5 shows the device 500 in an initial stage of being assembled. First the frame assembly 605 is located and retained in the bottom housing 604 via guide pins 901 or tabs that align with notches 902 in the frame 605. The notches on the frame are further aligned with ribs on the bottom housing, and pressed down in place. Depending on the exact type of polystyrene, plastic, or other resilient material used for the bottom housing, it is likely that the frame assembly 605 does not have to be firmly pressed into place as it just needs to be located and held in place with at least some friction. The port cover 601 is also snapped into one end of the bottom housing 604.

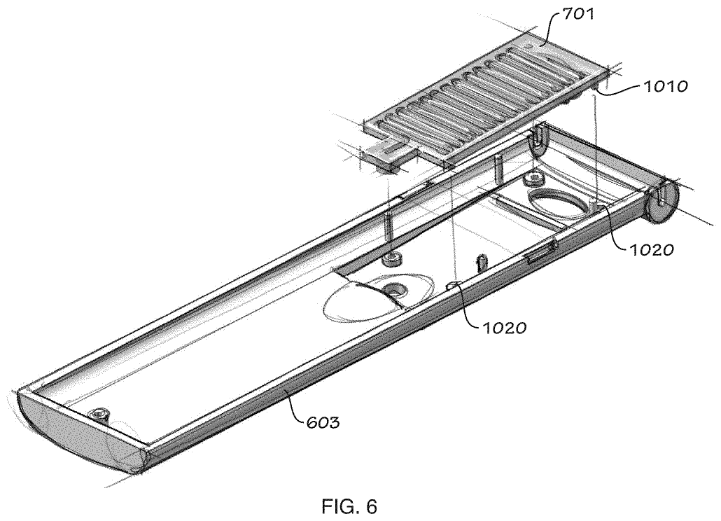

FIG. 6 shows the top housing 602 being assembled. The microfluidic chip 603 may be turned upside down. Four posts or pins 1010 on the microfluidic chip 603 may then be aligned with four hexagonal holes in the top housing. A press fit then holds these two parts together.

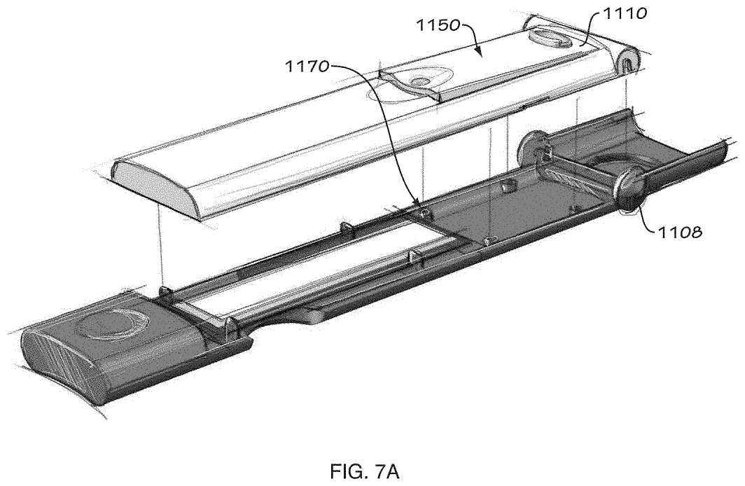

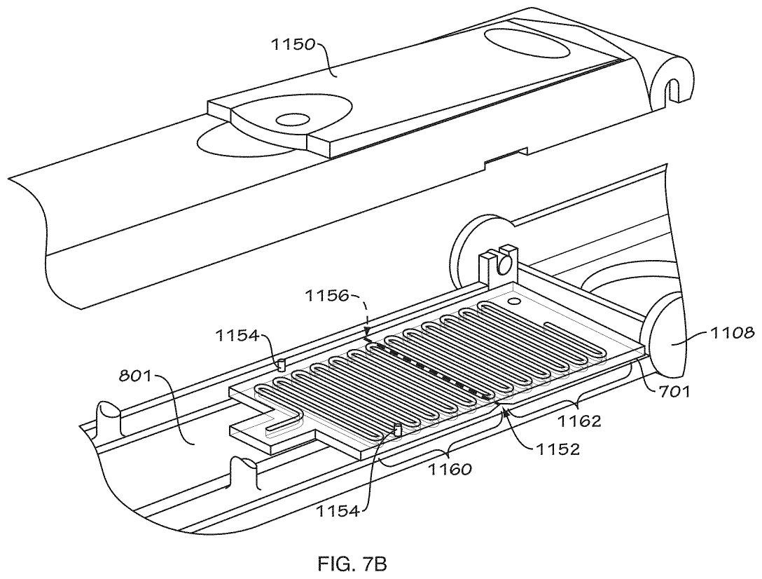

In FIG. 7A, the assembled top housing is rotated so that it is facing downward, and can now be pressed into place by engaging round pins of the top piece into hexagonal holes in the bottom piece. The pins and holes are designed to allow for tolerances within the molding process—a so-called Mattel™ fit.

It should be noted that the top housing 603 includes a thinned down area 1110, to provide a living hinged “door” 1150 that is activated after taking a sample.

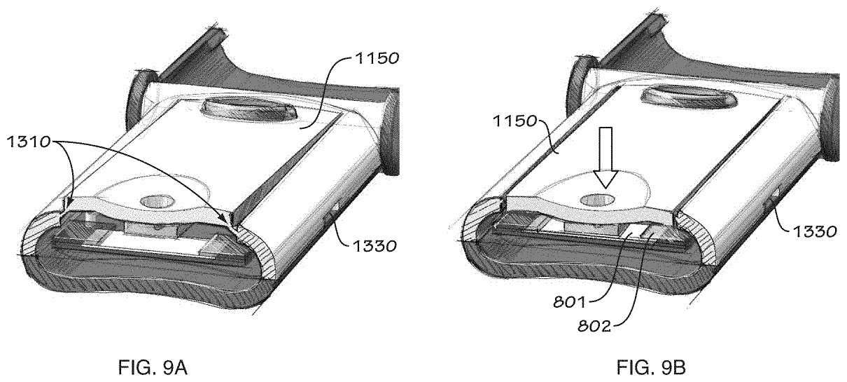

Referring briefly to the cross-sectional view of the top housing in FIG. 9A, two small bridges 1310 of plastic hold the hinged piece (or door) 1150 in place during assembly and before the sample is collected. Preferably formed of a styrene, the bridges 1310 can flex the door 1150 once or twice before they break, for example, after about a five-degree flex. A certain amount of force, such as a few pounds are needed to break the bridges 1310, permitting the hinged piece 1150 to snap down into a final horizontal position to close the device after use.

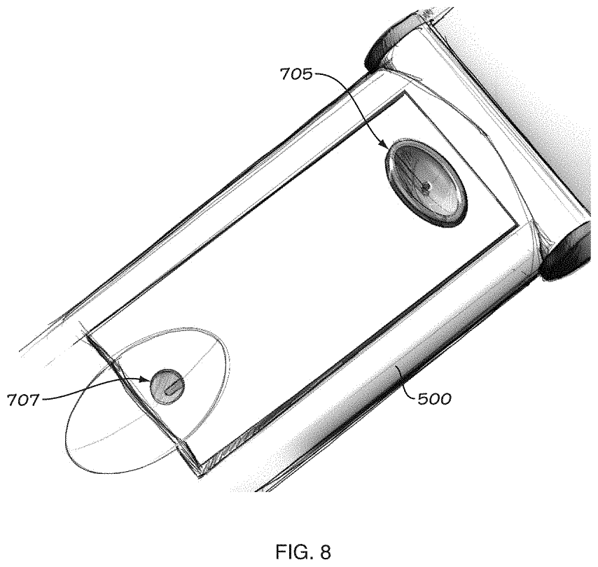

FIG. 8 shows how the device is used to collect a blood sample. The patient's (or other user's) pricked finger is scraped along an upper edge of the blood collection well 705 permitting blood to drip down into the port below. As the port is filled, the blood flows into the microfluidic chip (not shown in FIG. 8). When the blood passes through the channel(s) it eventually reaches the final window 707 that the user will see that they have collected enough blood for a complete sample.

In some implementations, additional windows may be placed between the sample port and the final window, so that the user can monitor the progress of collecting enough blood. Any or all such window(s) in the housing would preferably be flush with the outside surface of the device.

Returning attention to FIGS. 9A and 9B, after a blood sample is taken, the hinged piece 1150 is snap closed by the user. This snap closing action then also actually activates contact between the exit end of the microfluidic channels and the collection element 801. In other words, the end of the channel where the blood has reached is initially lifted off of and away from the collection media 801. However, pressing down on the door 1150 breaks the bridge pieces so that the sample port now comes in contact with the edge of the collection element 801, which then will start a wicking process of the blood into the collection element 801.

A slight interference fit may be provided by a slight bow in the frame pieces 802 and/or 803 supporting the collection element 801, to ensure good contact between the end of the microfluidic channel and the collection element 801. That's another reason why the frame is preferably open 805 at one end, so that the channels may come into direct contact with the collection element 801 to start the wicking process when the hinged door 1150 is closed.

With the sample port 705 being open on the other end, a vent on the back side (not shown) may be provided that encourages the blood to be completely wicked out of the microfluidic channels onto the collection paper.

FIG. 7B shows how the microfluidic subassembly (chip) 701 may have a thinned out portion 1152 or perforations 1156 in its substrate or channels. Protrusions 1154 also engage the inner surface of door 1150. Thus, when door 1150 is pushed down to activate the device, not only does the downward force on the door 1150 cause the chip 701 to come in contact with the collection element 801, but also the chip 701 now breaks. By closing the door and triggering the break, only a predetermined amount of the collected sample will now actually exit from the chip 701 onto the collection media 801. The amount of the sample taken is determined by the area of the microfluidic channels in section 1160 of the chip 701. Any excess collected by the microfluidics channels in section 1162 on the other side of the break (e.g, the section nearest the hinge) will not flow onto the collection element 801.

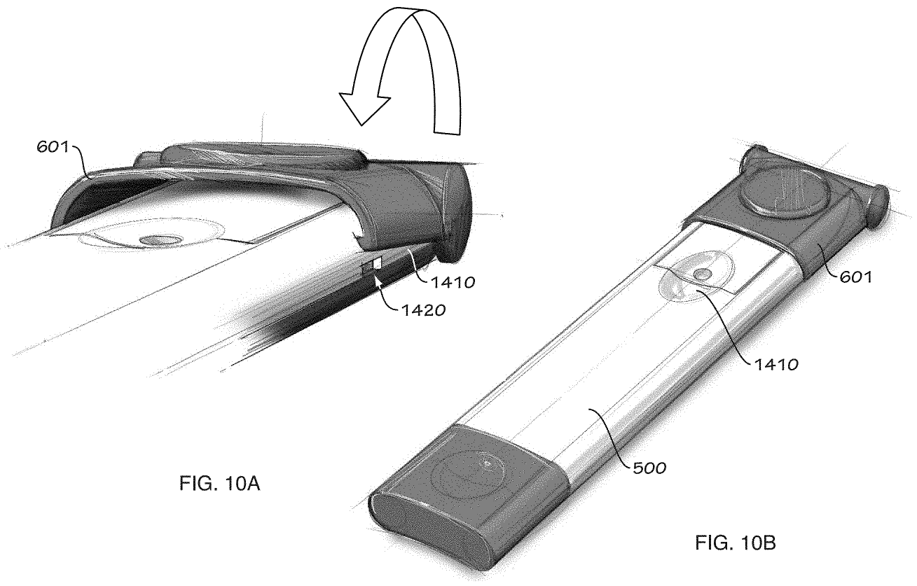

FIGS. 10A and 10B show how the hinged port cover 601 is snapped over (clasped or locked) after the sample is collected. This closes off any biohazard presented by the sample port before shipping. Pins in the cover and holes 1420 in the housing should be sized and shaped provide a one-time snap closure so that the sample port 705 is permanently captured and so that the cover 601 cannot easily be pulled off such as with a fingernail. This provides protection against accidental tampering, but not necessarily intentional tampering and so forth.

With the port cover 601 now closed, the cassette device 500 is fully contained and ready to be shipped to a remote laboratory such as within a pouch (not shown).

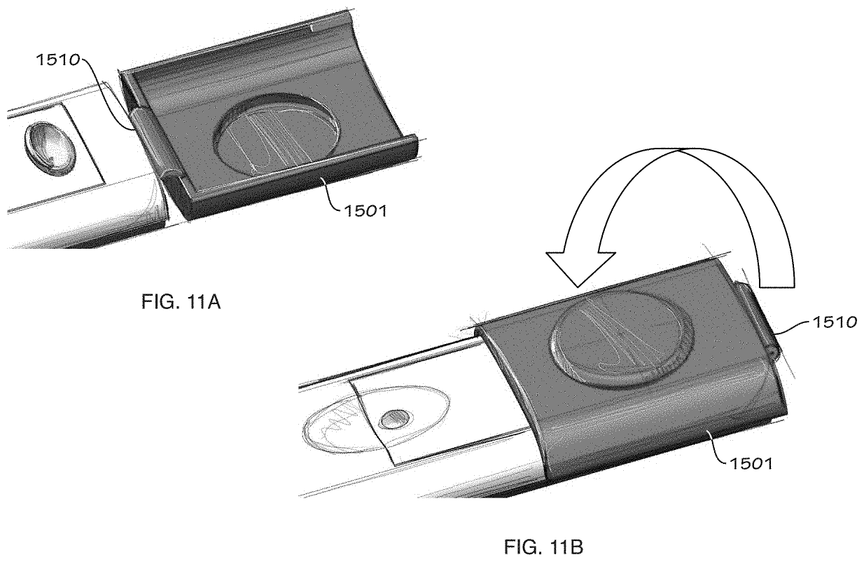

FIGS. 11A and 11B are an alternate design for the port cover 601. Here the port cover 1501 is a molded, living hinge on the bottom housing. The advantage of this arrangement is that it eliminates one extra part to mold and inventory and so forth. The disadvantage is that if the hinge between the port cover and the top housing is a little bit flexible or not perfectly flat when formed it might affect assembly.

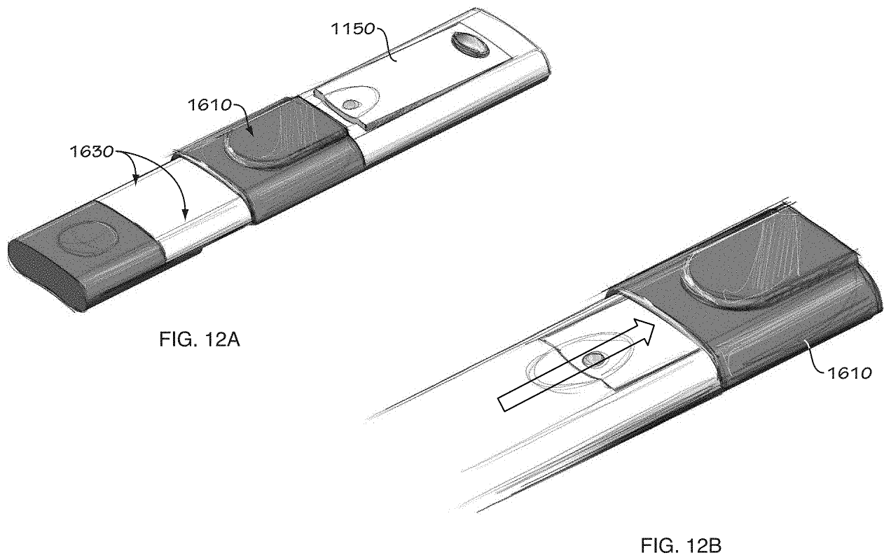

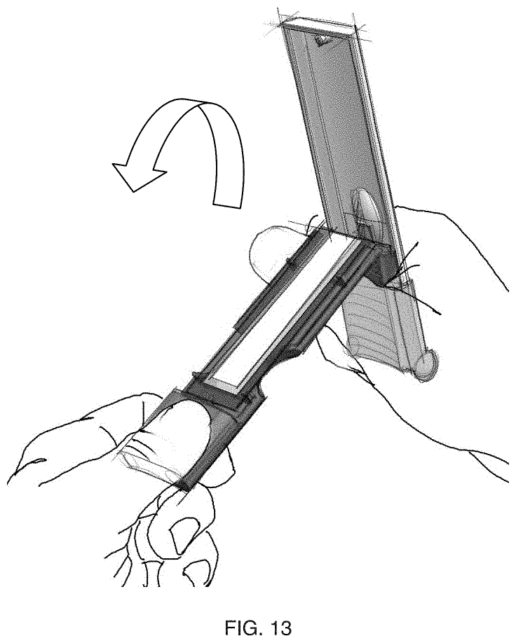

FIGS. 12A and 12B are another alternate design for the closure. This approach provides a sliding cover piece (or cap) 1610 is shaped to engage a line or ridge 1630 formed on or between the top and/or bottom housings. A detent or other feature inside the housing may hold sliding cover 1610 in place before the device is used. After the blood sample collection operation is completed, the user just simply slide the cap 1610 to the far edge, which in turn presses down on the door 1150 to cover and lock the port in one motion. That is, the user can grip the cassette 500 with say the left hand on the one end, and slide the cover towards the right-side end to close the cassette.

In other arrangements, the sliding cover 1610 can be designed to slide in the opposite direction, which may require extending the cassette a little bit to provide a surface area behind the collection port.

In other arrangements (not shown in the drawings) a twisting motion of the cap on a threaded housing can serve to activate the device and to sever the metered blood sample from the sample well. Twisting of the cap can also move the exit end of the microfluidic channel into contact with the absorbtive media by moving them closer in the vertical and/or horizontal axis. Some iterations may include the use of ramps in the housing to reliably reposition components.

In some implementations a cassette composed of the sample collection well and microfluidic chip can be a separate unit. The cassette is used to collect the blood sample and is then inserted into device. This may simplify the housing that holds the frame assembly as the insertion of the cassette would provide the activating motion to create fluid contact with the absorbtive media.

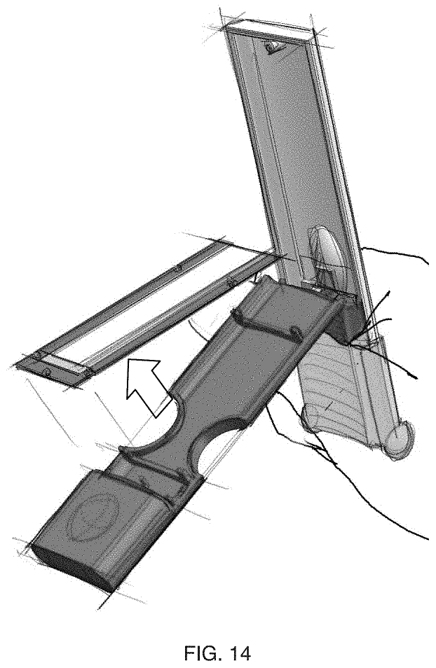

FIGS. 13 and 14 show how the device is disassembled at the laboratory. On one end of the cassette there was only a single pin engaging into a hexagonal hole 1320 (see FIGS. 9A and 9B), so the press fit is easily overcome to access the inside of the device. As shown in FIG. 13, one approach is for a lab technician to hold the device in their right hand, and pull down with the thumb of their left hand on the other end, to easily “peel” open the frame assembly 605 (similar to how a banana is peeled open) to now expose the collection paper. As per FIG. 14, they can now with the right hand dispose of the cassette while continuing to hold the frame in the left hand. The technician can then use a tool with the open right hand and punch one or more holes in the paper to collect a plasma-only portion of the sample, for example.

As shown, finger recesses in the frame and or cover piece assist with the disassembly process.

In addition, a bar code may be placed on the back side of the frame during manufacture and thus becomes visible at this final disassembly stage.

What is claimed is:

1. A blood sample collection device comprising:
a housing;
a collection well;
a metering component coupled to the collection well for collecting a metered portion of the blood sample;
absorptive media, disposed in a frame, adjacent to and in fluid communication with the metering component; and
a movable member engaging an upper region of the collection well, and providing push-driven mechanical force to initiate collection of a blood sample onto the absorptive media.

2. The device of claim 1 wherein a breakable tab is disposed between the movable member and the frame supporting the absorptive media.

3. The device of claim 2 wherein a hinged cover is disposed over the collection well.

4. The device of claim 1 wherein the the metering component is disposed on on a substrate composed of two components that separate upon application of the mechanical force.

5. The device of claim 1 wherein the well and metering component form a subassembly that is disposed adjacent the frame containing the absorbtive media.

6. The device of claim 1 additionally wherein the absorbtive media is a microfluidic separation media for separating plasma from whole blood in the blood sample.

7. The device of claim 6 additionally comprising one or more supports for supporting the microfluidic separation media within the housing.

8. The device of claim 1 additionally comprising:
an anticoagulant disposed within the metering component.

9. The device of claim 1 wherein the absorptive media is removable from the housing after application of the mechanical force.

10. A device for collecting a fluid sample comprising:
a sample port cover,
a top housing,
a fluid collection assembly,
a bottom housing, and
a frame assembly, wherein
the fluid collection assembly includes a sample port, an exit end, and one or more microfluidic channels disposed on a substrate, the microfluidic channels coupled to collect a fluid sample from the sample port via passive wicking action, and the substrate having at least one area of reduced dimension;
the frame assembly includes a storage element disposed between a top frame a bottom frame, with a least one end of either top frame or bottom frame being open and disposed adjacent but spaced away from the exit end of the fluid collection assembly, and the storage element comprising a storage media for storing the fluid sample;
the top housing including a door and an area of reduced thickness providing a living hinge for the door, such that pushing on the door causes the fluid communication between the exit end of the collection assembly and the storage element.
